(19) United States
(12) Patent Application Publication
(10) Pub. No.: US 2008/0071250 A1
(43) Pub. Date: Mar. 20, 2008

(54) Urinary tract catheter

(76) Inventor: William E. Crisp, Paradise Valley, AZ (US)

Correspondence Address: TOD R NISSLE, PO BOX 55630, PHOENIX, AZ, 85078, US

(21) Appl. No.: 11/985,401

(22) Filed: Nov. 14, 2007

Related U.S. Application Data
(63) Continuation-in-part of application No. 11/653,183, filed on Jan. 12, 2007.
Application No. 11/653,183 is a continuation of application No. 10/406,329, filed on Apr. 3, 2003, now abandoned.

(51) Int. Cl. A61M 27/00

(52) U.S. Cl. 604/544

(57) ABSTRACT

A urinary catheter is provided that reduces the likelihood of infection. The catheter includes a distal end positioned in a bladder. The distal end utilizes an inflated balloon that is positioned in the bladder to prevent removal of the distal end of the catheter from the bladder. The balloon, when inflated, contacts areas of the bladder that are spaced away from the urethral opening and from the trigone in the bladder. Urine drainage openings are formed in the distal end, are positioned laterally from the balloon, and can extend down to the inner surface of the bladder to facilitate drainage of urine from the bladder formed takes on a tapered configuration that reduces pressure on the trigone. The catheter incorporates one-way valves which function to make the catheter a close system that minimizes the risk of infection. Spaced apart concentrations of silver and zinc are incorporated on the inner and outer surfaces of the catheter to further reduce the risk of infection.

This is a continuation-in-part of application Ser. No. 11/653,183, filed Jan. 12, 2007, which is a continuation of application Ser. No. 10/406,329, filed Apr. 3, 2003, now abandoned.

This invention pertains to a catheter.

More particularly, the invention pertains to an indwelling urinary tract catheter system that minimizes the likelihood that an infection will occur in a patient as the result of insertion or subsequent operation of the catheter in the patient. The present indwelling catheters account for over 30% of hospital infections that occasionally proceed to renal disease and even death.

In a further respect, the invention pertains to an indwelling urinary tract catheter system that provides increased patient comfort when the catheter is inserted is inserted into the bladder and that provides increased patient tolerance to long term use.

In a male, the preprostatic urethra and proximal part of the prostatic urethra are lined with the urothelial type epithelium. This urothelial epithelium is continuous with linings of the seminal vesicles and with prostate ducts and bulbourethral gland ducts. The continuity of the urothelial epithelium with the seminal vesicle linings and ducts of the prostate and bulbourethral gland contributes to the spread of urinary tract infections when the mucous membrane barrier is compromised by a conventional catheter.

Below the openings of the ejaculatory ducts, the epithelium lining is a patchily pseudostratified or stratified columnar type. Such stratified epithelium lines the membranous urethra and most of the penile urethra. Mucous-secreting cells frequently occur in the stratified epithelium and there are numerous recesses connected to mucous glands.

The mucous membrane lining the female urethra comprises stratified epithelium and a supporting layer of connective tissue. The connective tissue is called the lamina propria and includes elastic fibers. The fibers are in an orientation extending both longitudinally and circularly around the urethra.

The internal urethral orifice opens into the inner bladder through the trigone. The ureters interconnect the bladder to the kidneys at the trigone. The trigone includes smooth muscle. The smooth muscle has two layers. The superficial muscle comprises the first layer. The deep detrussor muscle comprises the second layer. The deep trigonal muscle is the postero-inferior portion of the detrusor muscle. The superficial muscle is comprised of small diameter muscle bundles. These muscle bundles are continuous proximally with muscle bundles of the intramural ureters. The superficial muscle is thin but thickens at its superior border to form the interureteric crest. Comparable thickenings form along the lateral edges of the superficial muscle. The ureters interconnect the bladder and kidneys.

When a conventional catheter, for example a Foley catheter, is inserted into the bladder of an individual of either gender, the catheter extends upwardly through the urethra and the internal urethral orifice into the bladder. Openings are included for drainage in the outer tip of the distal end of the catheter for drainage. The distal end of the catheter includes balloon portion located beneath the openings. The opening functions to permit urine to drain from the bladder through a channel in the catheter and into a collection bag at the proximate end of the catheter. The balloon is inflated after the distal end is initially inserted through the urethra and the internal urethral orifice into the bladder. The inflated balloon seats on the trigone. Since the Foley catheter and other comparable catheters typically remain in and inhabit the bladder and urethra for extended periods of time, they are termed indwelling urinary catheters.

Indwelling urinary catheters account for over 30% of the morbidity in hospitals and nursing homes. Consequently, existing indwelling catheters dramatically increase the cost of medical care, and also increase for a patient the risk of illness and death.

One cause of infection by an indwelling catheter is the introduction into a patient of a microbe or other disease causing component during the insertion of the catheter into the urethra and bladder. The microbe typically exists on the external surface of the catheter due to improper sterilization, due to handling by the care giver, or due to other causes.

Another cause of infection by an indwelling catheter is the introduction into a patient of a microbe that ascends superiorly upwardly along the exterior surfaces of the catheter through the urethra and into the bladder, or that ascend along the inside surface of the catheter.

The other etiology of infection by an indwelling catheter is the introduction into a patient of a microbe through the channel that extends through the interior of the catheter and through which urine drains from the distal end of the catheter to the collection bag at the proximate end of the catheter.

Infections of the bladder can cause serious complications, especially for elderly patients, for patients having a weakened immune system, or for patients having preexisting diseases or weakened renal-bladder systems.

As noted, in the male urethra, the proximity of urothelial epithelium to the seminal vesicle epithelium and ducts of the prostate and bulbourethral gland contributes to the spread of urinary tract infections, and can result in infection of the prostate and testes.

Infections can also spread via the urethra to the bladder and on to the kidneys. If an infection originating in the bladder spreads to the kidneys, pyelonephritis can result. Pyelonephritis is inflammation of the kidney affecting the glomerulus or parenchyma.

Any infection in the lower urinary system has the potential of infecting the kidneys causing pyelonephritis which is a serious complication with potential dire consequences. Infection in the kidney (pyelonephritis) can cause nephrosclerosis with atrophy and renal failure (uremia) and/or hypertension.

Accordingly, it is highly desirable to provide a urinary tract catheter that reduces the possibility of infection.

Therefore, it is a principal object of the invention to provide an improved method and apparatus for removing urine from the bladder of a patient into a storage container with minimal risk for infection.

A further object of the invention is to provide an improved urine removal method and apparatus that reduces the likelihood that microbes or other disease causing components will be introduced into the body of a patient during catheter insertion and use.

These and other, further and more specific objects and advantages of the invention will be apparent from the following detailed description of the invention, taken in conjunction with the drawings, in which:

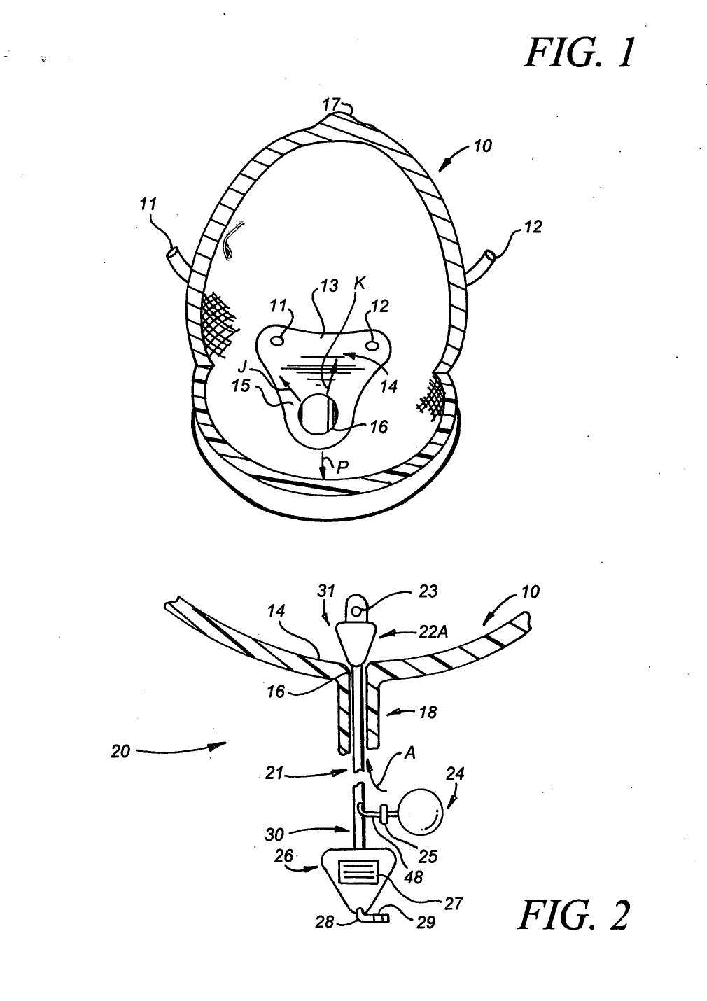

FIG. 1 is a front perspective partial section view illustrating a bladder;

FIG. 2 is a front elevation view illustrating a catheter constructed in accordance with the principles of the invention inserted in the urethra and bladder of a patient;

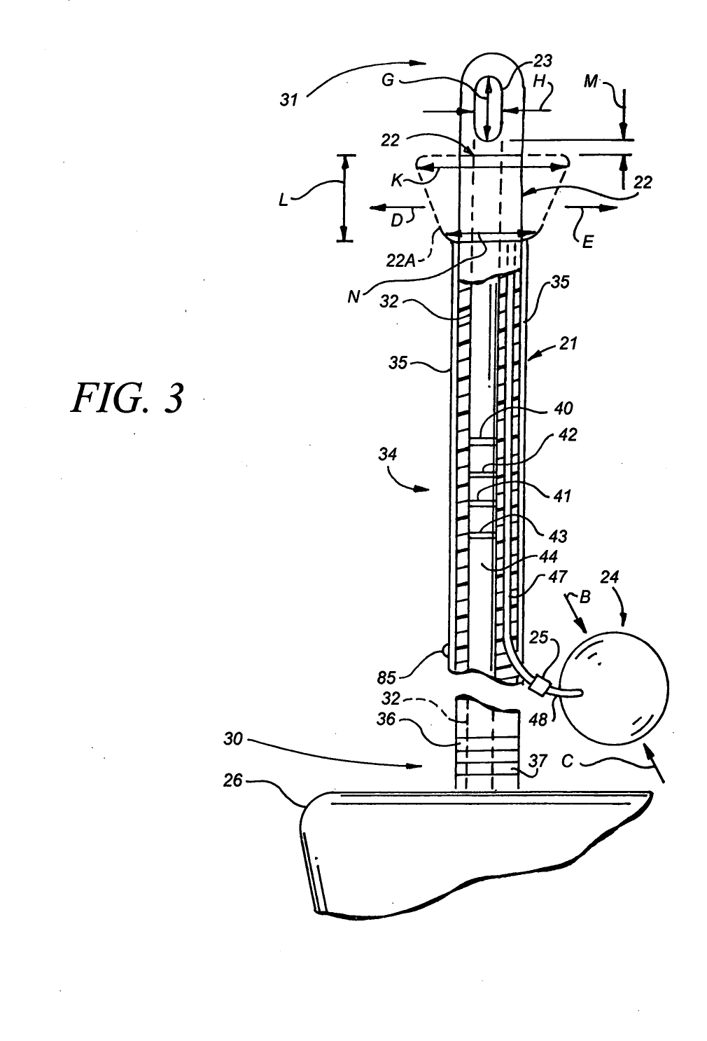

FIG. 3 is a front elevation view of the catheter of FIG. 2 illustrating additional construction details thereof;

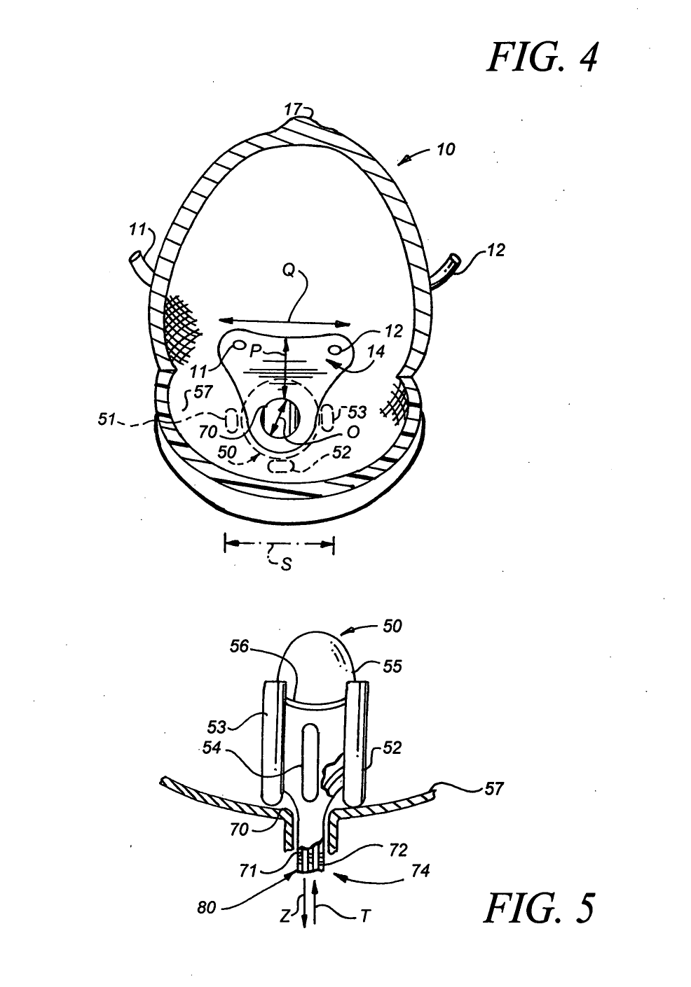

FIG. 4 is a front perspective partial section view illustrating a bladder and the use of an alternative embodiment of the invention therein;

FIG. 5 is a front elevation view illustrating an alternate embodiment of the catheter of the invention; and

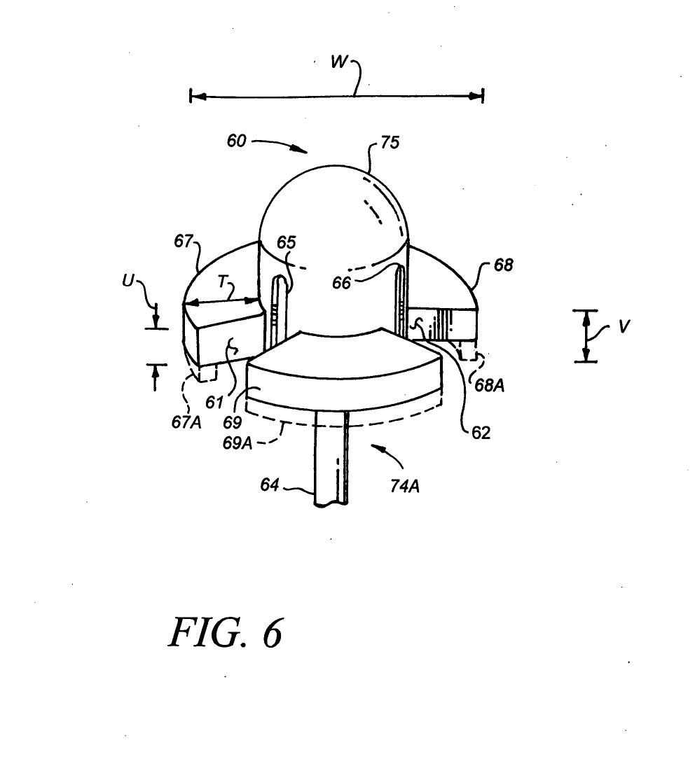

FIG. 6 is a perspective view illustrating still another embodiment of the catheter of the invention.

Briefly, in accordance with my invention, I provide an improved urinary catheter system for a bladder. The bladder includes a trigone. The catheter system comprises a urine collection container; an inflation system; and, an elongate pliable tube. The tube includes a proximate end extending to the urine collection container and located exterior of the urethra when the distal end is inserted in the bladder; includes a distal end shaped and dimensioned to be inserted through a urethra into the bladder and including an opening for draining urine from the bladder, and a balloon positioned beneath said opening and inflatable into a tapered configuration to minimize the contact surface area of the trigone; includes a first channel connecting the opening and the collection container; includes a second channel interconnecting the balloon and the inflation system such that the inflation system can be used to inflate the balloon into the tapered configuration; and, also includes an intermediate section positioned in the urethra when the distal end is inserted in the bladder.

In another embodiment of the invention, I provide an improved urinary catheter system for a bladder. The bladder includes a trigone. The improved catheter system comprising a urine collection container; an inflation system; and, an elongate pliable tube. The pliable tube has a proximate end extending to the urine collection container and located exterior of the urethra when the distal end is inserted in the bladder; has a distal end shaped and dimensioned to be inserted through a urethra into the bladder and including an opening for draining urine from the bladder, and an inflatable balloon positioned beneath the opening; has a first channel connecting the opening and the collection container; has a second channel interconnecting the balloon and the inflation system such that the inflation system can be used to inflate the balloon, and, has an intermediate section positioned in the urethra when the distal end is inserted in the bladder, the intermediate section including an outer foam layer.

In a further embodiment of the invention, I provide an improved urinary catheter system for a bladder. The bladder includes a trigone. The catheter system comprises a urine collection container; an inflation system; and, an elongate pliable tube. The pliable tube includes a proximate end extending to the urine collection container and located exterior of the urethra when the distal end is inserted in the bladder; includes a distal end shaped and dimensioned to be inserted through a urethra into the bladder and including an opening for draining urine from the bladder, and an inflatable balloon positioned beneath the opening; includes a first channel connecting the opening and the collection container; includes a second channel interconnecting the balloon and the inflation system such that the inflation system can be used to inflate the balloon; includes an intermediate section positioned in the urethra when the distal end is inserted in the bladder; includes at least one source of silver in the first channel; and, includes at least one source of zinc in the first channel and spaced apart from the source of silver.

In still another embodiment of the invention, I provide an improved urinary catheter system for a bladder. The bladder includes a trigone. The catheter system comprises a urine collection container; an inflation system; and, an elongate pliable tube. The tube has a proximate end extending to the urine collection container and located exterior of the urethra when the distal end is inserted in the bladder; has a distal end shaped and dimensioned to be inserted through a urethra into the bladder and including an opening for draining urine from the bladder, and an inflatable balloon positioned beneath the opening; has a first channel connecting the opening and the collection container; has a second channel interconnecting the balloon and the inflation system such that the inflation system can be used to inflate the balloon; has an intermediate section positioned in the urethra when the distal end is inserted in the bladder; has an exterior surface; has at least one source of silver on the exterior surface; and, has at least one source of zinc on the exterior surface spaced apart from the source of silver.

In still a further embodiment of the invention, I provide an improved urinary catheter system for a bladder. The bladder includes a trigone. The catheter system includes a urine collection container; an inflation system including a sealed reservoir of liquid; and, an elongate pliable tube. The elongate pliable tube has a proximate end extending to the urine collection container and located exterior of the urethra when the distal end is inserted in the bladder; has a distal end shaped and dimensioned to be inserted through a urethra into the bladder and including an opening for draining urine from the bladder, and an inflatable balloon positioned beneath the opening; has a first channel connecting the opening and the collection container; has a second channel interconnecting the balloon and the inflation system such that the inflation system can be used to inflate the balloon; and includes an intermediate section positioned in the urethra when the distal end is inserted in the bladder. The catheter system also includes apparatus for pressurizing the liquid to displace the liquid from the sealed reservoir through the second channel into the balloon to inflate the balloon.

In yet a further embodiment of the invention, I provide an improved urinary catheter system for a bladder. The bladder includes a trigone. The catheter system comprises a urine collection container; an inflation system; and, an elongate pliable tube. The pliable tube includes a proximate end extending to the urine collection container and located exterior of the urethra when the distal end is inserted in the bladder; includes a distal end shaped and dimensioned to be inserted through a urethra into the bladder and including an opening for draining urine from the bladder, and an inflatable balloon positioned beneath the opening; includes a first channel connecting the opening and the collection container; includes a second channel interconnecting the balloon and the inflation system such that the inflation system can be used to inflate the balloon; includes an intermediate section positioned in the urethra when the distal end is inserted in the bladder; and, includes an exterior self-lubricating surface.

In yet another embodiment of the invention, I provide an improved urinary catheter system for a bladder. The bladder includes a trigone. The catheter system comprises a urine collection container; an inflation system; and, an elongate pliable tube. The pliable tube has a proximate end extending to the urine collection container and located exterior of the urethra when the distal end is inserted in the bladder; has a distal end shaped and dimensioned to be inserted through a urethra into the bladder and including an opening for draining urine from the bladder, and an inflatable balloon positioned beneath the opening, the opening extending less than two centimeters above said balloon; includes a first channel connecting the opening and the collection container; includes a second channel interconnecting the balloon and the inflation system such that the inflation system can be used to inflate the balloon; and, includes an intermediate section positioned in the urethra when the distal end is inserted in the bladder.

In still yet a further embodiment of the invention, I provide an improved urinary catheter system for a bladder. The bladder includes a trigone. The catheter system comprises a urine collection container including a one-way valve for emptying the container; includes an inflation system; and, includes an elongate pliable tube. The tube includes a proximate end extending to the urine collection container and located exterior of the urethra when the distal end is inserted in the bladder; includes a distal end shaped and dimensioned to be inserted through a urethra into the bladder and including an opening for draining urine from the bladder, and an inflatable balloon positioned beneath the opening; includes a first channel connecting the opening and the collection container; includes a second channel interconnecting the balloon and the inflation system such that the inflation system can be used to inflate the balloon; and, includes an intermediate section positioned in the urethra when the distal end is inserted in the bladder.

In still yet another embodiment of the invention, I provide an improved urinary catheter system for a bladder. The bladder includes a trigone. The catheter system comprises a tapered urine collection container; an inflation system; and, an elongate pliable tube. The pliable tube includes a proximate end extending to the urine collection container and located exterior of the urethra when the distal end is inserted in the bladder; includes a distal end shaped and dimensioned to be inserted through a urethra into the bladder and including an opening for draining urine from the bladder, and a balloon positioned beneath the opening; includes a first channel connecting the opening and the collection container; includes a second channel interconnecting the balloon and the inflation system such that the inflation system can be used to inflate the balloon; and, includes an intermediate section positioned in the urethra when the distal end is inserted in the bladder.

In a further embodiment of the invention, I provide an improved urinary catheter system for a bladder. The bladder includes a trigone. The catheter system comprises a urine collection container including a light reflective scale; an inflation system; and, an elongate pliable tube. The pliable tube has a proximate end extending to the urine collection container and located exterior of the urethra when the distal end is inserted in the bladder; has a distal end shaped and dimensioned to be inserted through a urethra into a bladder and including an opening for draining urine from the bladder, a balloon positioned beneath the opening; has a first channel connecting the opening and the collection container; has a second channel interconnecting the balloon and the inflation system such that the inflation system can be used to inflate the balloon; and, has an intermediate section positioned in the urethra when the distal end is inserted in the bladder.

In another embodiment of the invention, I provide an improved urinary catheter system for a bladder. The bladder includes a trigone. The improved catheter system comprises a urine collection container; an inflation system; and, an elongate pliable tube. The tube has a proximate end extending to the urine collection container and located exterior of the urethra when the distal end is inserted in the bladder; a distal end shaped and dimensioned to be inserted through a urethra into a bladder and including an opening for draining urine from the bladder; a first channel connecting the opening and the collection bag; an intermediate section positioned in the urethra when the distal end is inserted in the bladder, the intermediate section including an exterior surface comprised of hydrophilic foam.

In still a further embodiment of the invention, I provide improvements in combination with a urinary tract. The urinary tract includes a bladder including a trigone, ureters, an internal wall surface, and an internal orifice, and includes a urethra, the urethra opening into and having a top located at the bladder. The improvements comprise a catheter system to drain urine from the bladder while minimizing the likelihood of infection and of discomfort to the urinary tract. The improvements comprise a urine collection container; and, an elongate pliable tube unit. The tube unit includes a distal end in the bladder. The distal end includes at least one inflated balloon contacting the internal wall surface of the bladder at points spaced apart from the top of the urethra, and at least one drainage opening positioned laterally from the balloon to drain urine from the bladder. The tube unit also includes a proximate end connected to the urine collection container; an intermediate section interconnecting the distal end and the proximate end and at least partially extending through the urethra; an inner surface; and, an outer surface. The balloon can contact the internal wall surface at points spaced apart both from the top of the urethra and the trigone. The drainage opening can extend points adjacent the interior wall surface of the bladder. At least one area of zinc and one area of silver spaced apart from the zinc area can be formed on the inner surface to create electrical current between the areas when the areas are connected by an electrically conductive liquid bridge. At least one area of zinc and one area of silver spaced apart from said zinc area can be formed on the outer surface to create electrical current between the areas when the areas are connected by an electrically conductive liquid bridge.

Turning now to the drawings, which depict the presently preferred embodiments of the invention for the purpose of illustrating the practice thereof and not by way of limitation of the scope of the invention, and in which like reference characters refer to corresponding elements throughout the several views, FIG. 1 illustrates a bladder, generally indicated by reference character 10. Bladder 10 includes ureters 11 and 12. Each ureter interconnects bladder 10 with a kidney (not shown). Trigone 14 is where the ureters 11 and 12 empty into the bladder and is adjacent the top of the urethra and the urethral orifice 16. The top of the urethra (i.e., the urethral wall) circumscribes the urethral orifice 16 and is intermediate the trigone and urethral orifice 16. Trigone 14 includes interureteric crest 13. Bladder 10 also includes uvula 15 and urethral orifice 16. The male urethra extends from the internal urethral orifice in the urinary bladder to the external urethral orifice at the end of the penis.

FIGS. 2 and 3 illustrate an indwelling urinary catheter system, generally indicated by reference character 20, inserted in the urethra 18 and bladder 10 of a patient. System 20 includes elongate pliable tube 21 fabricated from rubber or from some other polymer or other desired material. The distal end 31 of tube 21 include tapered, generally conically shaped inflated balloon 22A and opening 23. Urine drains from bladder 10 through opening 23 and through channel 32 into a urine collection bag 26 or other collection container. Proximate end 30 extends to bag 26. Bag 26 is tapered and, like inflated balloon 22A, generally has the shape of an inverted cone.

The tapered configuration of inflated balloon 22A is important in the practice of the invention because the lower portion of inflated balloon 22A contact and presses against a smaller area of the trigone than do the spherical balloons used in conventional urinary catheters. Further inflated balloon 22A has a height of less than two centimeters, preferably in the range of 0.5 centimeter to about 1.9 centimeters. This height is less than the inflated height of balloons found in conventional catheters, which conventional balloons typically have a height of two to three centimeters. The width of the top of inflated balloon 22A is indicated by arrows K in FIG. 3 and is preferably less than two centimeters. The width of the bottom of inflated balloon 22A is indicated by arrows N in FIG. 3 and is about equal to the diameter of drainage tube 21. While the diameter of tube 21 can vary as desired, such diameter presently is about six millimeters.

The height of uninflated balloon 22 is about equal to the height of inflated balloon 22A. This height presently is less than two centimeters. The top of balloon 22 and of inflated balloon 22A is, as indicated by arrows M in FIG. 3, about one-eighth of an inch below the bottom of opening 23. The distance of balloon 22 below the bottom of opening 23 can vary as desired, but preferably is in the range of one-eighth of an inch to one-quarter of inch.

The tapered configuration of collection bag 26 is important in the practice of the invention because the shape of bag 26 helps to insure that the bag drains completely when one-way valve 29 and tube 28 are utilized to drain bag 26 into another container. The collection bag 26 can have spaced apart concentrations of silver and zinc on the inside of the bag to prevent ascending infection. Urine drains from bag 26 through tube 28 and one-way valve 29 under gravity. A two-way valve or any other desired valve system can be incorporated in bag 26. A one-way valve is preferred, however, because it reduces the likelihood that microbes will enter bag 26 and travel up channel 32 and escape into the bladder 10 and urethra 18. Similarly, an inflation system other than the liquid filled resilient orb 24 can be utilized in the practice of the invention. Orb 24 is, however, preferred because it is sealed and reduces the likelihood microbes will travel up the catheter into the bladder 10.

The exterior surface 33 of tube 21 is preferably, but not necessarily, comprised of a self-lubricating or smooth material so that K-Y jelly or other lubricants are not necessary when tube 21 is inserted in the urethra and bladder of a patient, thereby avoiding another potential source of infection. By way of example, and not limitation, surface 33 can be comprised of teflon or polypropylene. A portion 34 of tube 21 intermediate the distal 31 and proximate 30 ends of tube 21 is preferably provided with an exterior hydrophilic foam coating 35 that softly engages the internal mucous membrane of urethra 18. Further, coating 35 preferably is impregnated, coated, or otherwise supplied with spaced apart concentrations of silver and zinc. The inside of tube 21 can also be impregnated, coated, or otherwise supplied with spaced apart concentrations of silver and zinc. The distance between these concentrations can vary as desired, but is typically in the range of 0.25 mm to 4.0 mm, preferably 0.5 to 1.5 mm. When coating 35 contacts bodily fluids, an electrical current arises between the silver and zinc concentrations. This current attracts microbes to the silver concentrations. When the microbes come into contact with the silver concentrations the microbes are killed.

All bacteria and other pathogenic organisms carry a negative charge. When the bimetallic pattern of dissimilar metals is moistened by body fluids (urine, mucous, etc.) A current flow is induced that draws the bacteria and other pathogenic organisms to the positively charged silver anode where the organisms are destroyed. The silver attaches to the sulfhydril groups and denatures the protein complexes vital to respiration and other functions.

In FIG. 3, a strip 36 of silver is shown spaced apart from a strip 37 of zinc on the exterior surface of tube 21. Similar strips can be formed on or in foam coating 35. Similar spaced apart strips of silver 40, 41 and zinc 42, 43 can be formed on the interior surface 44 of channel 32. The concentrations of silver and zinc can take on desired patterns or forms other than strips. For example, patterns of spaced apart dots, squares, etc. can be utilized. Metals other than silver and zinc can, if desired, be utilized to produce electrical current when tube 21 contacts bodily fluids. Silver and zinc are presently preferred because they produce a microbiocidal wet battery that does not promote the evolution of resistant organisms. Pathogenic organisms do not develop resistance to silver as they do to antibiotics.

The smooth outer surface of a conventional catheter flattens the inner surface of the urethra and makes it difficult for glands along the urethra to excrete moisture to the urethral lining. Further, natural movement of the urethra permits air to move into the urethra between the catheter tube and the urethral lining and to dry the mucosa. The foam coating 35 used in the catheter of the invention helps to avoid such problems and to avoid irritation of the urethral meatus. First, the foam coating absorbs moisture and swells. The moisture in the foam functions to moisturize the urethral lining to reduce the likelihood the mucosa will dry. Second, when the hydrophillic foam coating swells it prevents air from entering the urethra and drying the mucous membrane. The foam is also coated or impregnated with interspersed concentrations of silver and zinc for the prevention of infection. Third, since the surface of the foam is not continuous but comprises a plurality of openings circumscribed by wall structures (much like the hexagonal openings in a honeycomb are circumscribed by wall structures), the foam produces a plurality of point or edge contacts and permits portions of the mucosa to extend partially or completely into openings in the foam. This in essence increases the mucosa surface area contacted by the foam and facilitates the transfer of moisture from the foam to the mucosa or vice-versa. Fourth, the point-edge contact of the foam with the mucosa functions to form seals that further hinder the travel of air into the urethra intermediate tube 21 and the inner lining of the urethra. Coating 35 can consist of any hydrophilic foam or other material that produces the above-described edge or point contacts and includes pores that increase the surface area contact between the coating 35 and mucosa in the urethra. The foam is preferably, but not necessarily, resilient.

In use, a catheter system 20 is provided. The balloon 22 is not inflated. The exterior of tube 21 is self-lubricated, either because of the self-lubricating characteristics of the material used to fabricate tube 21, or because of a lubricating or friction reducing coating or material (i.e., Teflon) applied to the exterior of tube 21. Tube 21 is inserted through urethra 18 until distal end 31 is at the position illustrated in FIG. 2. Orb 24 is manually squeezed and compressed in the directions indicated by arrows B and C in FIG. 3, or apparatus other than a user's fingers is used, to force fluid through one-way valve 25 and through channel 47 in the direction of arrow A to fill (i.e., inflate) balloon 22 and cause it to expand in the directions of arrows D and E from the stored, uninflated configuration 22 depicted in FIG. 3 to the expanded, tapered configuration indicated by dashed lines 22A in FIG. 3 and illustrated in FIG. 2. When hollow orb 24 is compressed, a saline solution or other liquid in orb 24 is forced through one-way valve 25 and conduit 48 to channel 47. Valve 25 prevents liquid in balloon 22 or channel 47 from flowing back into orb 24. The use of orb 24 to hydraulically fill balloon 22 avoids requiring a nurse to fill balloon 22 in conventional fashion with a syringe. The use of such a syringe is a frequent cause of infection.

After balloon 22 is inflated to its expanded, tapered configuration 22A, installation of the catheter system 20 is complete. Urine that accumulates in bladder 10 flows through opening 23 and channel 32 into bag 26. Bag 26 is periodically emptied using conduit 28 and one-way valve 29. The catheter is removed by first cutting the catheter to release the pressurized fluid from the inflated balloon 22A, causing the balloon to deflate. The catheter is then carefully removed from the urethra.

The shape and dimension of opening 23 can vary as desired. It is presently preferred however, that opening 23 be about five millimeters high, as indicated by arrows G, and three millimeters wide, as indicated by arrows H. A conventional urinary catheter includes an opening that is five millimeters high and two and one-half millimeters wide. The addition width H in the catheter system of the invention is important in reducing the likelihood that opening 23 will be blocked.

Graduated markings 27 can be provided on bag 26 or on any other urine collecting container utilized in the invention. The markings 27 indicated the volume of urine in bag 26 or indicate what proportion of bag 26 is filled or remains to be filled. Markings 27 can be light reflective to facilitate readings the markings. Markings 27 can also fluoresce. The catheter of the invention can be fabricated without balloon 22, orb 24, and channel 47. Either this “balloon-less” catheter or a catheter of the type illustrated in the drawings can be used to drain an abscess or remove fluid from other body cavities.

The shape and dimension of balloon 22 and inflated balloon 22A can vary as desired. However, one significant advantage of the balloon 22 of the invention is that when it is inflated, the lower portion of balloon 22 preferably rests and seats on top of the urethra without contacting the trigone, or, only contacting a relatively small portion of the trigone. The lower portion of the inflated balloon 22A can contact both the top of the urethra and the trigone. The lower portion of the inflated balloon 22A can also contact or extend adjacent a portion of the upper inner surface of the urethral wall that is adjacent the top of the urethra. The area of the trigone contacted by the bottom of the balloon is preferably less than the area of the top of the urethra contacted by the bottom of the balloon, or, is preferably less than the sum total of the surface area of the top of the urethra and of the upper inner surface of the urethral wall contacted by the bottom of the balloon. In another embodiment of the invention, the area of the trigone contacted by the bottom of the balloon is (1) 50% of the sum total of the surface area of the top of the urethra contacted by the bottom of the balloon 22A and the surface area of the trigone contacted by the bottom of the balloon, or, (2) 50% of the sum total of the surface area of the top of the urethra contracted by the bottom of the balloon 22A, of the surface area of the upper inner surface of the urethral wall contacted by the bottom of the balloon 22A, and, of the surface area of the trigone contacted by the bottom of the balloon. Most preferably, the bottom of the inflated balloon 22A normally does not contact the trigone at all.

In another embodiment of the invention, the surface area of the top of the urethra and/or of the trigone that is contacted by the bottom of the inflated balloon and is adjacent and extends outwardly from the perimeter of internal orifice 16 in any direction, as indicated by arrows J and K and P, extends outwardly no more than one millimeter away from the perimeter of the internal orifice 16. Consequently, since the top of the urethral wall that circumscribes orifice 16 may be at least one millimeter wide, if the surface area of tissue around orifice 16 that is contacted by the bottom of the inflated balloon extends outwardly from orifice 16 a distance of one millimeter or less, then the balloon may not contact any portion of the trigone.

In a further embodiment of the invention, the surface area of the top of the urethra and/or of the trigone that is contacted by the bottom of the inflated balloon and is adjacent and extends outwardly from the perimeter of internal orifice 16 in any direction, as suggested by arrows J and K and P, extends outwardly no more than two millimeters away from the perimeter of the internal orifice 16. Consequently, since the top of the urethral wall that circumscribes orifice 16 may be at least two millimeters wide, if the surface area of tissue around orifice 16 that is contacted by the bottom of the inflated balloon extends outwardly from orifice 16 a distance of two millimeters or less, then the balloon may not contact any portion of the trigone.

In still a further embodiment of the invention, the surface area of the top of the urethra and/or of the trigone that is contacted by the bottom of the inflated balloon and is adjacent and extends outwardly from the perimeter of internal orifice 16 in any direction, as suggested by arrows J and K and P, extends outwardly no more than three millimeters away from the perimeter of the internal orifice 16.

In still another further embodiment of the invention, the surface area of the top of the urethra and/or of the trigone that is contacted by the bottom of the inflated balloon and is adjacent and extends outwardly from internal orifice 16 in any direction, as suggested by arrows J and K and P, extends outwardly no more than four millimeters away from the perimeter of the internal orifice 16.

In yet a further embodiment of the invention, the surface area of the top of the urethra and/or of the trigone that is contacted by the bottom of the inflated balloon and is adjacent and extends outwardly from internal orifice 16 in any direction, as suggested by arrows J and K and P, extends outwardly no more than five millimeters away from the perimeter of the internal orifice 16.

In yet another embodiment of the invention, the surface area of the top of the urethra and/or of the trigone that is contacted by the bottom of the inflated balloon and is adjacent and extends outwardly from internal orifice 16 in any direction, as suggested by arrows J and K and P, extends outwardly no more than six millimeters away from the perimeter of the internal orifice 16.

In yet still a further embodiment of the invention, the surface area of the top of the urethra and/or of the trigone that is contacted by the bottom of the inflated balloon and is adjacent and extends outwardly from internal orifice 16 in any direction, as suggested by arrows J and K and P, extends outwardly no more than seven millimeters away from the perimeter of the internal orifice 16.

In yet still other embodiments of the invention, the surface area of the top of the urethra and/or of the trigone that is contacted by the bottom of the inflated balloon and is adjacent and extends outwardly from internal orifice 16 in any direction, as suggested by arrows J and K and P, extends outwardly no more than eight, nine, ten, eleven, twelve, thirteen, fourteen, fifteen, sixteen, seventeen, eighteen, nineteen or twenty millimeters away from the perimeter of the internal orifice 16, as the case may be and as may be desirable depending on the physiology of the individual.

The lower portion of inflated balloon 22A can be seated on top of the urethra because the urethra is normally contracted at least in part and because the top of the urethra therefore forms a support surface or area on which the lower portion of balloon 22A can rest. Such a support surface exists even when tube 21 is in the urethra. The support surface circumscribes tube 21.

The use and construction of an alternate catheter design is illustrated in FIGS. 4 and 5. Catheter of FIG. 5, as does the catheter illustrated in FIGS. 2 and 3, has two primary objectives, 1) to decrease catheter associated infection, and 2) to provide an anatomic design improvement to decrease discomfort the patient and for more complete emptying of the bladder.

The catheter of FIG. 5 (as well as the catheter of FIG. 6) is, in the manner illustrated in FIGS. 2 and 3 for the catheter earlier described herein, preferably, but not necessarily, constructed utilizing a double lumen drainage tube utilizing a lubricius polymer. The drainage tube is coated with a biocompatible copolymer both inside and outside the drainage tube.

The copolymer coating holds micron sized silver and zinc particles that create a protective antimicrobial barrier.

The metals are held in a biocompatible copolymer that erodes to lactic acid when the catheter is moistened by urine or other biological liquids and when, consequently, a galvanic current is created.

All pathogens that infect the bladder—including the resistant staph infections (MRSA)—carry a negative charge. The galvanic current directs the pathogens to the positive silver anode where they are destroyed. The galvanic current has no effect on the urethra or bladder function and is not perceived by the patient.

The insertion technique of our catheter does not require a complicated apparatus as described by Bidwell in U.S. Pat. No. 3,053,257.

One type of known retention catheter is the Foley catheter. The Foley catheter consists of a double lumen tube that stays in the bladder held by a balloon. The double lumen tube include a first tube through which urine is drained and includes a second smaller tube. The Foley catheter includes a balloon that is inflated via the smaller tube. The distal end of the catheter has an opening which allows urine to drain into a collection bag. The balloon on a Foley catheter is inflated by using a syringe to force a liquid to inflate the balloon. A valve is provided to maintain the liquid under pressure so that the balloon remains inflated.

The balloon on the new catheter of the invention does not require a syringe to inflate the balloon (a major source of infection). The sterile fluid is held by a closed system reservoir that is manually squeezed by digital pressure to inflate the balloon by simple hydraulics.

The discomfort created by an indwelling catheter is primarily related to a dry urethral mucosa that is caused by the catheter (especially rubber catheters) compressing the urethral mucosa against the muscular wall of the urethra. The urethral mucosa adheres to the catheter, causing discomfort when the patient moves.

The exterior of the catheter illustrated in FIG. 5 is coated with silver and zinc micron particles that produce increased secretion of mucous from the urethra thereby avoiding the discomfort of an adherent catheter. The increased mucous along with the bi-metallic galvanic current creates another barrier to ascending infection.

The other cause of discomfort is pressure of the retention balloon resting on the trigone of the bladder.

The ureters and proximal end of the urethra enter the bladder at the trigone. The trigone contains the majority of the nerves in the bladder and is extremely sensitive to any pressure such as the pressure caused by the standard inflated balloon of the Foley Catheter.

The catheter of FIG. 5 is held in place by a small inflated feet 51, 52, 53 so that there is minimal or no contact with the trigone and such that the likelihood that the bladder wall and will herniate into the urethral portion of the catheter is significantly reduced.

The catheter balloon described by in the Deford reference (U.S. Pat. No. 5,304,214) is much larger and has greater contact with the trigone and bladder wall because its function is to stabilize the urethral bladder junction while the prostate is being treated.

In the catheter of FIG. 5, the urine drainage openings 54 are each interposed between spaced apart pair of inflated feet 51, 52, 53 and extend between feet 51 to 53. Openings 54 can be spaced apart from or adjacent a foot 51 to 53. In contrast, in a Foley catheter, the drainage openings are located above the inflated balloon. Preferably, although not necessarily, when the catheter of FIG. 5 is inserted in the manner illustrated in FIG. 5, openings 54 extend downwardly to a point near inner surface 57 of the bladder to allow a more complete emptying of the bladder thereby eliminating residual urine which is another source of infection.

The lower end of the new catheter of the invention is attached to a portable drainage bag that is triangular in shape for easier emptying. Fluorescent numbers can be included on the bag make it easier for reading fluid volumes. A one way valve prevents reflux.

The entire system of the catheter drainage tubing and collection bag is a single unit, thereby providing further protection from contamination and infection.

In FIG. 4, trigone 14 typically has a maximum width indicated by arrows Q of about twelve to thirteen millimeters (mm) (in an adult of average size), and the distance from the top 70 of the urethra to the top of the trigone is indicated by arrows P and typically is about eight mm. The diameter of the top 70 of the urethra is indicated by arrows O and typically is about four mm; however, the urethra dilates to eight to ten mm. As can be seen in FIG. 4, the trigone 14 extends substantially completely around the top 70 of the urethra. Ureters 11 and 12 extend outwardly from the trigone 14 of bladder 10. The presently preferred width of the distal end 50 of the catheter illustrated in FIGS. 4 and 5 (and of the catheter illustrated in FIG. 6 and other similar catheters constructed in accordance with the invention) is indicated by arrows S and is about eight mm, but the shape and dimensioned of the distal end 50 and other components of the catheter can vary as desired. A width of eight mm is preferred because such a width typically enables a balloon 51 to 53, 67 to 69 to contact the interior surface 57 of bladder 10 at points that are, in the manner illustrated in FIG. 4 by dashed lines 51 to 53, outside trigone 14 and spaced apart from the top 70 of the urethra.

The distal end 50 of the tube unit 74 catheter is more particularly illustrated in FIG. 5 and includes hollow base or housing 55. The shape and dimension of base 55 can vary as desired. In FIG. 5, base 55 has a generally cylindrical shape with a rounded, hemispherical top. Base 55 can be fabricated from any desired material, but typically presently comprises a substantially rigid polymer. Urine drainage openings 54 are formed in base 55 and lead to a drainage channel 71 in tube unit 74 that permits urine to drain downwardly in the directly of arrow Z to a urine collection container. A smaller channel 72 extending through the tube unit 74 and base 55 to balloons 51 to 53 is used to inflate balloons 51 to 53 to the configurations illustrated in FIG. 5. A hollow liquid filled orb (not shown in FIG. 5) similar to orb 24 is squeezed to cause a fluid, preferably a liquid, to flow under pressure from the orb, up through channel 72, and into balloons 51 to 53 to inflate the balloons. The balloons 51 to 53 can, if desired, be interconnected by channels 56 such that liquid from one balloon 51 to 53 can flow into one or both of the other balloons.

As depicted in FIG. 4 by dashed lines 51 to 53, when the distal end 50 of the tube unit 74 is positioned inside bladder 10 and balloons 51 to 53 are inflated, the foot, or bottom, of each balloon contacts the inner surface 57 of bladder 10 at points outside of and spaced apart from trigone 14. While the foot of each balloon 51 to 53 can, if desired, contact the trigone 14, it is preferred that each balloon not contact the trigone 14.

Further, as can be seen in FIG. 5, each urine drainage opening 54 is preferably positioned laterally from a balloon 52, 53 and extends downwardly. The distance that an opening 54 extends downwardly can vary as desired, but it is preferred that at least one opening formed in distal end 50 extend to a point even with or adjacent the inner surface 57 of bladder 10. Such location of drainage opening 54 laterally from a balloon 52, 32 and extending downwardly toward and/or flush with inner surface 57 improves drainage of urine from bladder 10 and significantly reduces the likelihood of infection.

FIG. 6 illustrates an alternate embodiment of a tube unit 74A constructed in accordance with the invention. Tube unit 74A is illustrated in FIG. 6 in the configuration it takes on when positioned in a bladder 10. The hollow base 75 of the distal end 60 includes drainage openings 65 and 66 formed therein, along with three spaced apart pie-shaped inflated balloons 67 to 69. The shape and dimension of base 75 is comparable to that of base 55 in FIG. 5. Balloons 67 to 69 can be inflated in any desired manner, as can the balloons on other embodiments of the invention illustrated herein. However, a liquid filled, manually compressed orb 24 of the type earlier described is preferred to direct liquid up from the proximate end (not shown) of tube 64 through a channel in tube 64 to balloons 67 to 69. A space 61, 62 is formed between each pair 67-69, 69-68, 68-67 of balloons 67 to 69. Urine is able to drain from bladder 10, into spaces 61 and 62, into drainage openings 65 and 66, and hence into a channel in tube 64 that carries urine to a urine collection container (not shown) attached to the proximate end (not shown) of tube 64.

The balloons 67 to 69 in distal end 60 in FIG. 6 extend outwardly from base 75 and likely would contact the inner surface 57 of bladder 10 at points adjacent the top 70 of the urethra. While this configuration can be utilized, more preferred is to form balloons 67 to 69 to each include a peripheral foot 67A to 69A, respectively, that functions to contact inner surface 57 at points spaces apart from the top 70 of the urethra, functions to lift the remaining portions of balloons off surface 57, functions to space the remaining portions away from surface 57, and functions to minimize the area of the trigone 14 contacted by a balloon 67 to 69.

When feet 67A to 69A are utilized, balloons 67 to 69 can, if desired, be shaped and dimensioned to be interconnected to form a continuous “doughnut” balloon that extends completely around base 75. Feet 67A to 69A extend downwardly from the “doughnut” balloon and against the inner surface 14 of the bladder and function to maintain the “doughnut” balloon positioned above and spaced apart from the inner surface 14 of the bladder. In this configuration, each drainage opening 65, 66 extends from a point beneath the “doughnut” balloon downwardly to the inner surface 14 of the bladder. Each drainage opening extends downwardly from the bottom of the doughnut balloon a distance that is about equal to the distance that feet 67A to 69A extend downwardly from the bottom of the doughnut balloon. Utilizing drainage openings 65, 66 that extend downwardly toward surface 14 from between balloons 67, 68 in the manner illustrated in FIG. 6, or that extend downwardly toward surface 14 from beneath a “doughnut” balloon or other balloon, is important in the practice of the invention because such drainage openings facilitate complete drainage of urine from the bladder, particularly when openings 65, 66 extend to surface 14 of the bladder. Facilitating complete drainage of urine from the bladder is important because it reduces the likelihood of infection.

The preferred width, indicated by arrows W, of the distal end of tube unit 74A (or tube unit 74) is typically at least about eight mm, although such can vary. The preferred width, indicated by arrow T, of a balloon 67 to 69 (or 52 to 53) is typically at least two mm, although such can vary. The preferred height, indicated by arrows U or V, of a balloon 67 to 69 (or 52, 53) is in the range of two to six mm, although such can vary.

In use, when a distal end 50, 60 is inserted through a urethra and into a bladder, the balloons 52, 53, 67 to 69 are not inflated; otherwise it would not, as is appreciated by those of skill in the art, be physically possible to insert a distal end 50, 60 through the urethra into a bladder. The balloons 52, 53, 67 to 69 are inflated only after a distal end 50, 60 is positioned in a bladder 10.

A dimple or other mark 85 (FIG. 3) can be included on a tube 80, 64 so that medical personnel can judge the orientation of a distal end 50, 60 as it is inserted through the urethra into a bladder 10, and, can (if possible) position end 50, 60 such that balloons 51 to 53 rest on inner surface 57 and do not rest on trigone 14 after balloons 51 to 53 are inflated while in bladder 10.

Having described my invention in such terms as to enable those of skill in the art to make and practice it, and having described the presently preferred embodiments thereof, I claim:

1. In combination with a urinary tract including
a bladder including
a trigone,
ureters,
an internal wall surface, and
an internal orifice, and
a urethra, the urethra opening into and having a top located at the bladder, the improvements comprising a catheter system to drain urine from the bladder while minimizing the likelihood of infection and of discomfort to the urinary tract, said improvements comprising
(a) a urine collection container;
(b) an elongate pliable tube unit having
(i) a distal end in the bladder and including
at least one inflated balloon contacting the internal wall surface of the bladder at points spaced apart from the top of the urethra, and
at least one drainage opening positioned laterally from said balloon to drain urine from the bladder,
(ii) a proximate end connected to said urine collection container,
(iii) an intermediate section interconnecting said distal end and said proximate end and at least partially extending through the urethra,
(iv) an inner surface, and
(v) an outer surface.

2. The combination of claim 1 wherein said balloon contacts the internal wall surface at points spaced apart both from the top of the urethra and the trigone.

3. The combination of claim 1 wherein said drainage opening extends to points adjacent the interior wall surface of the bladder to facilitate complete drainage of urine from the bladder.

4. The combination of claim 1 including at least one area of zinc and one area of silver spaced apart from said zinc area on said inner surface to create electrical current between said areas when said areas are connected by an electrically conductive liquid bridge.

5. The combination of claim 1 including at least one area of zinc and one area of silver spaced apart from said zinc area to create electrical current between said areas when said areas connected by an electrically conductive liquid bridge.

6. In combination with a urinary tract including
a bladder including
a trigone,
ureters,
an internal wall surface, and
an internal orifice, and
a urethra, the urethra opening into and having a top located at the bladder, the improvements comprising a catheter system to drain urine from the bladder while minimizing the likelihood of infection and of discomfort to the urinary tract, said improvements comprising
(a) a urine collection container;
(b) an elongate pliable tube unit having
(i) a distal end in the bladder and including
at least one inflated balloon contacting the internal wall surface of the bladder at points spaced apart from the top of the urethra, and
at least one drainage opening extending downwardly from said balloon to drain urine from the bladder,
(ii) a proximate end connected to said urine collection container,
(iii) an intermediate section interconnecting said distal end and said proximate
end and at least partially extending through the urethra,
(iv) an inner surface, and
(v) an outer surface.

7. The combination of claim 6 wherein said balloon contacts the internal wall surface at points spaced apart both from the top of the urethra and the trigone.

8. The combination of claim 6 wherein said drainage opening extends to points adjacent the interior wall surface of the bladder to facilitate complete drainage of urine from the bladder.

9. The combination of claim 6 including at least one area of zinc and one area of silver spaced apart from said zinc area on said inner surface to create electrical current between said areas when said areas are connected by an electrically conductive liquid bridge.

10. The combination of claim 1 including at least one area of zinc and one area of silver spaced apart from said zinc area to create electrical current between said areas when said areas connected by an electrically conductive liquid bridge.
